(19) United States
(12) Patent Application Publication
(10) Pub. No.: US 2008/0009773 A1
(43) Pub. Date: Jan. 10, 2008

(54) Mathematical Modeling System for assisting practitioners in the detection of global subluxations, segment subluxations and their correlation - postural/spinal coupling

(76) Inventors: Donald Dean Harrison, Evanston, WY (US); Sanghak Oh Harrison, Evanston, WY (US); Tadeusz J. Janik, Huntsville, AL (US)

Correspondence Address: Dr. Donald Harrison, D.C., Ph.D., M.S.E., P.O.Box 1590, Evanston, WY, 82931, US

(21) Appl. No.: 11/456,338

(22) Filed: Jul. 10, 2006

(51) Int. Cl. A61B 5/103

(52) U.S. Cl. 600/595

(57) ABSTRACT

The present invention is a method of modeling the biomechanics of the body using three mathematical models: (1) one model detects global subluxations (postural), (2) another model detects segment subluxations (spinal), and (3) the third mathematical model correlates the results of the global subluxations analysis and segment subluxations analysis—postural and spinal coupling.
a) Based on a mathematical model used to identify global/postural subluxations from the adjustments to the positioning of a scalable digital model over the digital images of the lateral, posterior and anterior views of a patient.
b) Based on a second mathematical model and specific views of spinal X-rays, used to identify segmental subluxations from the adjustments to the positioning of a scalable digital model over the patient's spine.
c) Using a third mathematical model to assist in the correlation the global and segment subluxations.
d) Using these models alone, or in combination, the individual is given suggested linked mirror-image exercises.

### BRIEF DESCRIPTION

The present invention relates to a system and method for invention used to assist the practitioner in the detection of global subluxations (postural) and segment subluxations (spinal) and their correlation—postural and spinal coupling, based on mathematical models.

Specifically the method uses the following steps alone or in combination:
Part 1: Modeling the biomechanics of the body using a mathematical model that is superimposed on the patient's body to assist in detecting global subluxations (postural).
Part 2: Mathematically modeling the vertebrae of the spine by superimposing a scalable digital model over the individual's spine to assist in detecting segment Subluxations (spinal).
Part 3: Correlating the results of the positioning of global subluxations to the segment subluxations of the spine from the use of a third mathematical model (postural and spinal coupling).

### BRIEF DESCRIPTION OF ILLUSTRATIONS

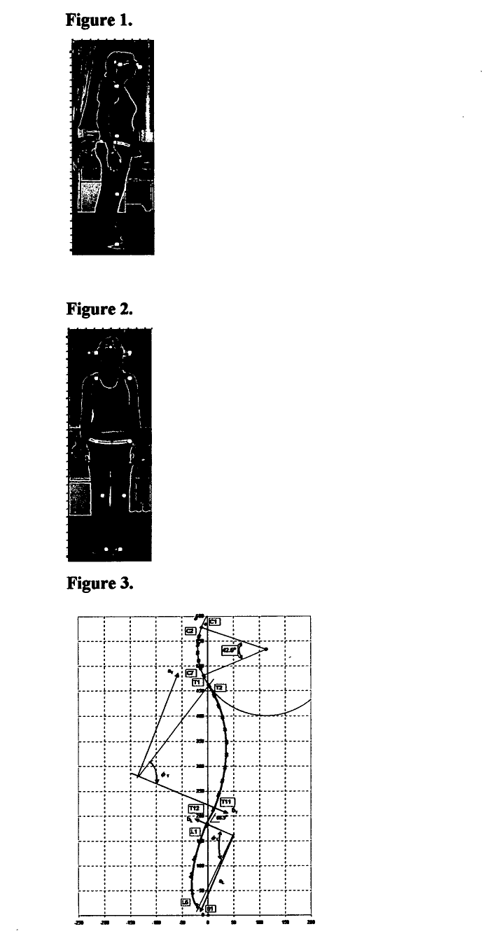

FIG. 1. Model overlay on photograph are taken with the right side to the camera

FIG. 2. Model overlay on photographs are taken of the subject from the front

FIG. 3. CBP® Full-spine Normal Model that is superimposed on the x-rays of the spine.

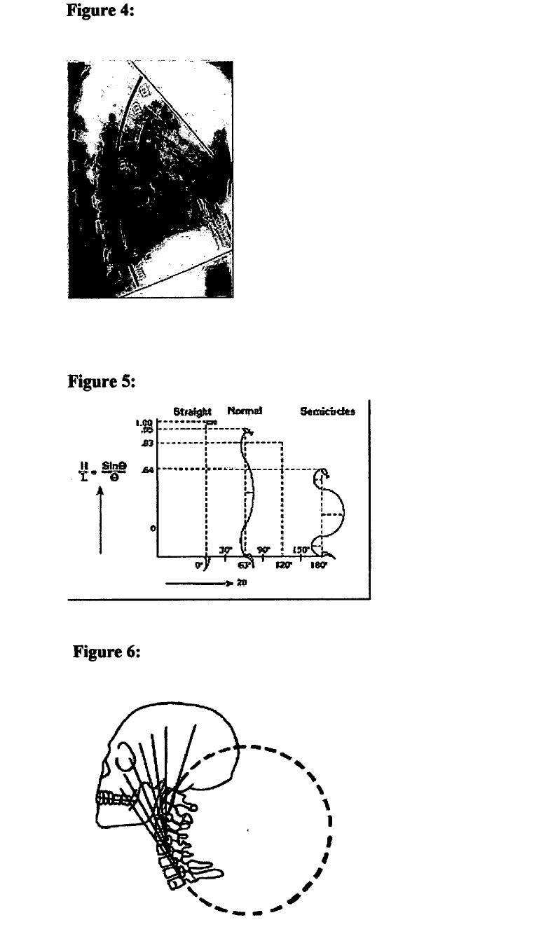

FIG. 4. X-Ray and Template Overlay

FIG. 5. 1979 Harrison Spinal Model

FIG. 6. 1996 CBP® C1-T1 Cervical Model

FIG. 7. 1998 CBP® Lumbar Model

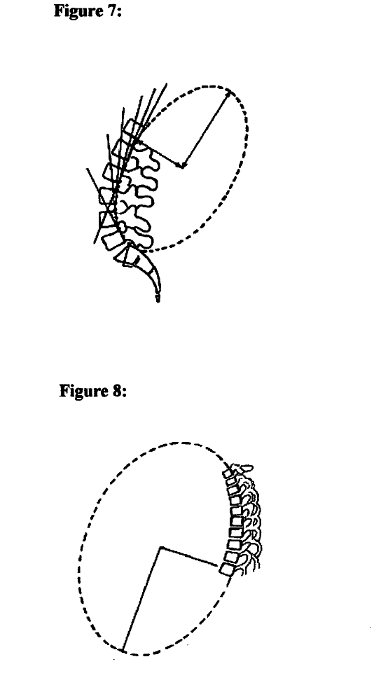

FIG. 8. 2002¹³ & 2003¹⁴ CBP® Thoracic Models

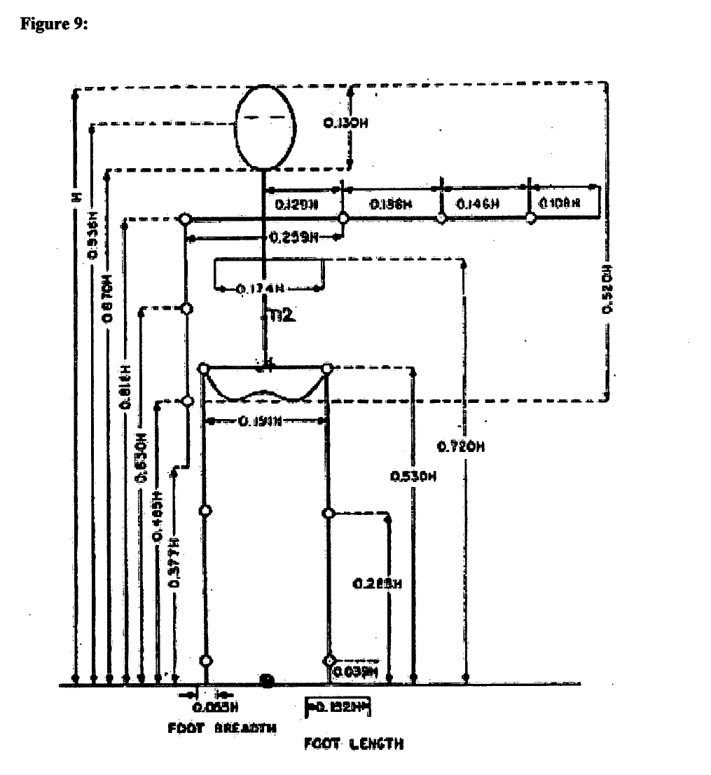

FIG. 9. Dempster's Body Segment Parameter Data for 2-D Studies.

### BACKGROUND OF THE INVENTION

### Description of Prior Art

In simplest terms, a subluxation is when one or more of the vertebrae of your spine move out of position and create pressure on, or irritate spinal nerves. Spinal nerves are the nerves that come out from between each of the bones in your spine. This pressure or irritation on the nerves then causes those nerves to malfunction and interfere with the signals traveling over those nerves.

Subluxations are really a combination of changes going on at the same time. These changes occur both in your spine and throughout your body. For this reason vertebral subluxations as referred to as the “Vertebral Subluxation Complex”, or “VSC” for short.

In the VSC, various things are happening inside a body simultaneously. These various changes, known as “components,” are all part of the vertebral subluxation complex. Chiropractors commonly recognize five categories of components present in the VSC. These five are:
The Osseous (bone) Component is where the vertebrae are either out of position, not moving properly, or are undergoing physical changes such as degeneration.
The Nerve Component is the malfunctioning of the nerve. Research has shown that only a small amount of pressure on spinal nerves can have a profound impact on the function of the nerves.
The Muscle Component is also involved. Since the muscles help hold the vertebrae in place, and since nerves control the muscles themselves, muscles are an integral part of any VSC.
The Soft Tissue Component is when you have misaligned vertebrae and pressure on nerves resulting in changes in the surrounding soft tissues. This means the tendons, ligaments, blood supply, and other tissues undergo changes. These changes can occur at the point of the VSC or far away at some end point of the affected nerves.
The Chemical Component is when all these components of the VSC are acting on your body, and therefore causing some degree of chemical changes. These chemical changes can be slight or massive depending on what parts of your body are affected by your subluxations.

There are many different types of spinal models in the scientific literature. In 1987, Yoganandan et al.¹ grouped spinal models into the following four categories:
Geometrical Considerations,
Force Considerations,
Type of Analysis,
Applications of the Model.

In 2004 the CBP® Ideal Spinal Model, a Geometrical Considerations model, was finalized after many years of research and validation. The mathematical models included in this invention are bases on the CBP® Ideal Spinal Model and Dempster's Body Segment Parameter Data for 2-D Studies from D. A. Winter, Biomechanics and Motor Control of Human Movement, Second edition. John Wiley & Sons, Inc., Toronto, 1990.

Today practitioners do not have an automated system that uses an evidence-based mathematical model to assess for either global or segment subluxations, even less a system that correlates the both of them as in the postural-spinal coupling model.

There are multitudes of existing stand-alone and Web-based system that identify postural deviations. However, there are no systems today that will assist practitioners in identifying global and segment subluxations.

Furthermore, there are no existing systems that will automatically use mathematical models that are superimposed on the patient's body to assist in detecting global subluxations (postural), segment subluxations and the correlation of both to produce personalized assessments and mirror-imaged exercise regimens.

In addition, there is no way for a practitioner to re-evaluate the patient and quantify improvements in either or both the global or segment subluxations. This invention will also provide that assistance to the practitioner.

### SUMMARY OF THE INVENTION

One objective of the present invention is to provide a process and system to acquire vertebral positioning data to assist in the detection of segmental and regional subluxations using segmental angles, global angles, and translational distances (posterior tangents & modified Risser-Ferguson line drawing as CD, RZ, LD, LS angles on AP X-rays).

Another objective of the present invention is to provide a process and system to acquire positioning data of the head, rib cage, and pelvis as rotations and translations to assist in the detection of global subluxations.

Another objective of the present invention is to provide a process and system to assist in the correlation of the segment subluxations with the global subluxation.

Another objective of the present invention is to use the global subluxation analysis, the segment subluxation analysis and the postural-spinal coupling model to assist practitioners in the use of mirror image® methods (adjusting maneuvers and exercises).

Another objective of the present invention is to use the CBP® Ideal Spinal Model and digital photographs of spinal X-rays to identify segmental angles, global angles, and translational distances by analyzing the differences between the digital representation of the CBP® Ideal Spinal Model and the X-Rays.

Another objective of the present invention is to determine the 3-D position of the subject's head, rib cage, and pelvis from 2-D digital photographs by adjusting the starting digital representation of the mathematical model to adhesive strips on the patient's body and analyzing these differences Vs the vertical and horizontal plumb lines.

Another objective of the present invention is to provide a process and system which superimposes digital representations of the mathematical models for both the global subluxations and segment subluxations.

Another objective of the present invention is to provide a process and system which assists the practitioner in identifying the severity of both the global subluxations and segment subluxations by comparing the angles and distances obtained in the segment and global subluxation analysis to published normal values in the Index Medicus literature.

As such, the Mathematical Modeling System for assisting practitioners in the detection of global subluxations, segment subluxations and their correlation (postural-spinal coupling) radically changes the practice of biomechanical modeling analysis by creating an objective methodology and innovative technology.

This unique system is outlined in more detail below.

### DETAILED DESCRIPTION OF THE INVENTION & ILLUSTRATIONS

1. Modeling the biomechanics of the body using a mathematical model that is superimposed on the patient's body to assist in detecting global Subluxations (postural) by the analysis of head, rib cage, and pelvis postures in three-dimensions (3D) as rotations and translations along the three X, Y, and Z axes.
   The following steps are involved in modeling the biomechanics of the body using a mathematical model that is superimposed on the patient's body to assist in detecting global Subluxations (postural).
   Taking both left and right side and front view digital images of a patient. Please note below, an example containing some, but not all, of these images. (FIGS. 1 and 2).
   Vertically cropping the digital images in the different views to the head and feet of the patient to be able to accurately calibrate the scalable digital mathematical model. The scalable mathematical model is based on Dempster's Body Segment Parameter Data for 2-D Studies as described in Appendix 2.
   The horizontal digital mathematical model is automatically calibrated and scaled based on one of two methods:
      A horizontal ruler attached to the wall and/or
      The known horizontal length of the CBP® eye gear
   Automatically superimposing mathematical model which is scalable (based on the individual's height and body type) over the different views. Please note below, an example containing some, but not all, of these images, (See FIGS. 1 and 2.)
   Manually adjusting the computer generated digital mathematical model, as required, by dragging the end points of the scalable model lines over adhesive strips that were placed on the patient's body segments or CBP® eye gear.
   Creating a plumb line by using the scalable digital model to calculate plumb line in the vertical and horizontal planes.
      For the vertical plumb line the mathematical model calculates plumb as:
         For the lateral views from the middle of the ankle
         For the anterior and posterior views from the mid-point of the ankle level digital model
      For the horizontal model the mathematical model calculates plumb as:
         To the middle of the foot at ankle level
         Behind the knees
         At the T12 area for the torso line
         At the shoulders (AC joint)
         At the head, align the yellow circles to the white points on the glasses
   Assist in identifying the global subluxations (postural) and creating an impact assessment and suggest a personalized mirror-imaged exercise routine based on the results.
   By determining the 3-D position of the subject's head, rib cage, and pelvis from 2-D digital photographs by manually adjusting the starting digital representation of the mathematical model to the patient's body and calculating the angular and distance differences Vs the vertical and horizontal plumb lines.
   The calculations for each of the mathematical model points is as follows: (See Table 1)
   Creating an impact assessment which indicates, but is not limited to:
      The global subluxations identified by the previous process.
      The severity of the global subluxation by a coding system as described below:
         Minor—green: any global subluxation that is less than 2 millimeters or 1 degree from plumb.
         Moderate—yellow: any global subluxation that is greater than 2 millimeters and less than 5 millimeters or greater than 1 degree and less than 3 degrees from plumb.
         Major—red: any global subluxation that is 5 millimeters or more or 3 degree or more from plumb.
      The impact of the global subluxations on the different effective weights of the subject's head, rib cage, and pelvis.
      Which of the mirror image® methods (adjusting maneuvers and exercises) a practitioner should use and in what time frame. These suggestions to the practitioner may vary from week to week.
         These mirror image® methods (adjusting maneuvers and exercises) are the exact opposite position (or in difficult cases, these may be in a more stressed position) of the patient's initial presenting global subluxation.
2. A method to assist in the detection of segment Subluxations (spinal) and their relative severity in a patient by using the CBP® Ideal Spinal Model and digital photographs of spinal X-rays to identify segmental angles, global angles, and translational distances by analyzing the differences between the digital representation of the CBP® Ideal Spinal Model and the X-Rays. A full description of the CBP® Ideal Spinal Model is available in Appendix 1. This method is comprised of the following steps:
   2.1. Loading digital images of a patient's spinal X-rays, including but not limited to:
      2.1.1. AP cervical or AP cervico-thoracic
      2.1.2. AP nasium
      2.1.3. AP full spine
      2.1.4. AP lumbo-pelvis
      2.1.5. AP Ferguson lumbo-pelvis
      2.1.6. AP femur head short leg
      2.1.7. lateral cervical
      2.1.8. lateral thoracic
      2.1.9. lateral lumbar
      2.1.10. lateral full spine
      2.1.11. cervical flexion & extension
      2.1.12. lumbar flexion & extension
   2.2. Superimposing a digital mathematical model of the vertebrae of the spine to find the specific points on the digital X-ray images. An example of this model follows: (See FIG. 3)
   2.3. Manually adjusting the computer generated digital mathematical spinal model, as required, by moving the lines of the model.
   2.4. Analyzing the points using the digital mathematical model of the vertebrae to arrive at angles and distances using posterior tangents & Modified Risser-Ferguson line drawing as CD, RZ, LD, LS angles on the X-rays.
      2.4.1. The process for the Cervical model is as follows:
         2.4.1.1. Move the dot on the Vertical axis line (VAL) with one end at T1 posterior-inferior body corner
         2.4.1.2. Move the other dot directly vertical from the last dot drawn in step 1 at the height of the posterior-superior lateral mass of C1
         2.4.1.3. The system will measure translation of the head on the z-axis
         2.4.1.4. Fit the curve that best fits between the dots drawn in steps 1 and 2. Keep the template completely vertical and now drag the red ideal curve from C1-T1
         2.4.1.5. If T1 is not visible, you may use C7.
         2.4.1.6. You would now just fit the VAL from C2-C7 instead of C1-T1 and use a smaller arc.
      2.4.2. The process for the Thoracic model is as follows:
         2.4.2.1. Locate and place a dot at posterior-inferior T11
         2.4.2.2. Fit the Vertical Axis Line (VAL) from posterior-inferior T11 until it passes through the T1 level;
         2.4.2.3. Locate the inferior-posterior corner of T2.
         2.4.2.4. Move the dot and drag the horizontal line through this dot until it intersects the VAL.
         2.4.2.5. Rotate the thoracic template model on the radiograph by rotating it until ends of the cut out curves as vertical
         2.4.2.6. Slide the template model until a “best fit” ellipse is found that will have one cut out end (check the numbers at each end for symmetry) containing the dot at posterior-inferior T11.
         2.4.2.7. While the TOP end will NOT be at superior-posterior T2, but rather the T2 inferior disc radial line will be placed where the horizontal line (step 3) and the VAL (step 2) meet.
         2.4.2.8. Drag the RED line along the cut out curve for this best fit ellipse from inferior-posterior T11 to SUPERIOR-POSTERIOR T2; Draw BLACK LINES along the posterior vertebral margins of T2 through T11. Please see example below: (See FIG. 4)
      2.4.3. The process for the Lumbar model is as follows:
         2.4.3.1. With the lateral lumbo-pelvic drag the vertical line (VAL) from the posterior-inferior body corner of S1 (parallel to vertical edge of digital image).
         2.4.3.2. Drag the horizontal line through the posterior-inferior body corner of T12.
         2.4.3.3. Locate the red line on the elliptical curve model for the posterior-superior sacral base. Drag this point to the patient's posterior-superior sacral base.
         2.4.3.4. Pivot the digital model until the top of the chosen curve intersects VAL.
         2.4.3.5. Measure the distance from this intersection with VAL to the intersection of the T12 horizontal line with VAL.
         2.4.3.6. Does the above procedure with the next curve model. Determine the distance that is minimum, several curves may be tried. The curve with the minimum distance to the intersection of the T12 horizontal line and VAL is the Best Fit Curve.
         2.4.3.7. Using this cut out Best Fit Curve in place from sacral base to VAL, drag the “RED” line through the cut out area from S1 to the height of T12.
         2.4.3.8. Drag the black x-ray model line draw along George's line from S1 to T12.
   2.5. The angles and distances are compared to published normal values in the Index Medicus literature.
   2.6. An assessment report is generated for the practitioner based on the results which includes:
      2.6.1. A section on diagnostic codes frequently used by healthcare providers when determining the state of degeneration of a patient's spine.
      2.6.2. There will be check boxes next to listed items in tables.
      2.6.3. The practitioner will choose the relevant codes and these will be printed in the assessment.
      2.6.4. In order to provide a service to the practitioner for follow-up evaluations, there will be a “Comparative” assessment, in which previous patient measurements will be compared to new/post measurements. These can be used by the practitioners to substantiate the care given to a patient and to decide the need for any further care.
3. A method to assist with correlating the results global subluxations assessment and segment subluxations assessment (postural and spinal coupling).
   3.1. Using the postural and spinal coupling model to further assist the practitioner in identifying global subluxations (postural) and segment subluxations (spinal). This includes:
      3.1.1. Using the results from the segment subluxation analysis for the following X-rays:
         3.1.1.1. AP cervical
         3.1.1.2. AP lumbo-pelvis
         3.1.1.3. Lateral cervical
         3.1.1.4. Lateral thoracic
         3.1.1.5. Lateral lumbar
      3.1.2. Validating the measurements in degrees and millimeters for the following views Vs the results obtained in 3.1.1:
         3.1.2.1. Tx head from the AP cervical view
         3.1.2.2. Tx thorax from the AP lumbar view
         3.1.2.3. Tz head from the lateral cervical region
         3.1.2.4. Tz thorax from the lateral lumbar-thoracic regions
         3.1.2.5. Rx head from Chamberlian's line on lateral cervical
         3.1.2.6. Rx thorax from lateral thoracic region (Lt or lateral full spine)
         3.1.2.7. Rx pelvis from lateral lumbo-pelvis
         3.1.2.8. Rz Pelvis from sacral HB angle on AP lumbar
         3.1.2.9. Rz thorax comparing to horizontal & then to HB angle
   3.2. Assist the practitioner in refining the results from the global subluxation analysis to be in line with the segment subluxation analysis.
   3.3. Aiding the practitioner in further refining the personalized mirror-imaged exercise routine, mirror-imaged adjustments on a drop table and mirror-imaged traction.
   3.4. An assessment report is generated for the practitioner, including:
      3.4.1. Impact on the patient's global subluxation assessment
      3.4.2. Modification to the mirror image® methods (adjusting maneuvers and exercises) a practitioner should use and in what time frame. These suggestions to the practitioner may vary from week to week.

While the invention has been described in this claim, it will be understood that it is capable of further modification and this application is intended to cover any variations, uses, or adaptations of the invention following, in general, the principles of the invention and including such departures from the present disclosure as come within known or customary practice within the art to which the invention pertains and as may be applied to the essential features here in before set forth, and as follows in the scope of the appended claims.

### DETAILED DESCRIPTION OF ILLUSTRATIONS/FIGURES

FIG. 1 illustrates the height dependent model overlay on one of the side view photographs of a subject. FIG. 2 illustrates this model overlay on the front view photograph of a subject. FIG. 3 illustrates the new CBP® Full-spine Normal Model that is superimposed on the x-rays of the spine to provide vertebral body corners for the User to click and drag to their proper locations. This model is the path of the posterior longitudinal ligament through the posterior body margins and is composed of separate ellipses in the different spinal regions (cervicals, thoracics, & lumbars). It has near perfect sagittal balance of vertical alignment of C1-T1-T12-S1. The sagittal curves have points of inflection (mathematic term for change in direction from concavity to convexity) at inferior of T1 and inferior of T12. FIG. 4 illustrates one of the Normal spinal curve templates, the thoracic template, placed over a side view x-ray of the thoracic spine (there are templates for the cervical and lumbar spines). FIG. 5 illustrates the old 1979 Harrison Spinal Model that was a Height-to-Length ratio based on two assumptions: (1) the spinal curvatures are arcs of circles and (2) the Delmas Index is ideal (H/L=0.95). FIG. 6 depicts the 1996 CBP® C1-T1 Cervical Model that was an arc of a circle.⁴ FIG. 6 provided an “average normal” model based on 400 subjects and an “ideal normal” model based on several hypothetical assumptions. It reported average and ideal normal values for each segmental angle (C2-3, C3-4, C4-5, C5-6, and C6-7) and a normal value for the global angle between posterior tangents on C2 and C7. FIG. 7 illustrates the 1998 CBP® Lumbar Model that was an arc of an ellipse, with b/a=0.4.¹¹ This Figure was derived from an “average normal” model based on 50 subjects and an “ideal normal” model based on several hypothetical assumptions. It reported average and ideal normal values for each segmental angle (T12-L1, L1-2, L2-3, L34, L4-5, and L5-S1) and an ideal value for the global angle between posterior tangents on L1 and L5. FIG. 8 The 2002¹³ & 2003¹⁴ CBP® Thoracic Models were arcs of ellipses, with b/a=0.7. These provided an “average normal” model based on 80 subjects and an “ideal normal” model based on several hypothetical assumptions. It reported average and ideal normal values for each segmental angle (T1-2, T2-3, T3-4, T4-5, T5-6, T6-7, T7-8, T8-9, T9-10, T10-11, and T11-12) and a normal value for the global angle between posterior tangents from T1-T12, T2-T11, and T3-T10. FIG. 9 Dempster's (USA Air Force study) Body Segment Parameter Data were suggested for 2-D Studies.

### Appendix 1—The CBP® Ideal Spinal Model

In 1979 Dr. Don Harrison used two major assumptions (and several smaller assumptions) to derive a sagittal spinal model.²⁻⁴ These were (1) all three spinal regions (cervical lordosis, thoracic kyphosis, & lumbar lordosis) are arcs of circles, and (2) the Delmas⁵ Height to Length ratio, H/L=0.95 index is ideal for the each region of the sagittal spine. Using some geometry and trigonometry, he arrived at the equation H/L=(sin θ)/θ=0.95, which when solved for 2θ provided a 63° arc for each spinal region, e.g., C1-T1 (FIG. 5).

Prior to 1979, there were others,6,7 who used the same major assumption of arcs of circles for the spinal curvatures, but with different second assumptions. In 1908, Goetz⁶ assumed that the radius of curvature (R) was equal to the length of the arcs (L), yielding 57.3° arcs, while in 1974, Pettibon⁷ assumed that the radius (R) equaled the chord of the arc (C), yielding 60° arcs. Table 1 compares these early spinal models. For years my father thought that he had done something special with his “different” 1979 spinal model, but looking back at the models in Table 1, it can be observed that all three of these models are included in the range of 57°-63° and would differ very little clinically, i.e., segmental angles of curvature (C2-3, C3-4, C4-5, C5-6, C6-7) and/or global angles of curvature from C2 to C7. (See Table 2)

In 1993 Dr. Don Harrison and Dr. Tad Janik determined an average model for the cervical model. From measurements on 400 lateral cervical radiographs from Dwight DeGeorge's clinic in Saugus, Mass., average segmental angles (C2-C7), global angles between C2 and C7, H/L, and anterior head weight bearing were obtained. These were compared to Dr. Harrison's old model of H/L=(sin θ)/θ, but with out forcing the exact value of 0.95 for normal. Dr. Harrison's old model predicted the average values within a mean error of 5%. This supported the assumption that the cervical spine was approximately a piece of a circle (arc of a circle); see FIG. 6. This was published in 1996.⁴

The measurements on sagittal spinal radiographs are made with posterior body tangents. This method of radiographic line drawing analysis has been reported to be highly reliable.⁸⁻¹⁰

Subsequently, Dr. Harrison and Dr. Janik worked on the lumbar spine. Out of several geometric choices (circle, hyperbola, parabola, sine wave, etc), they decided use an ellipse. After trial and error, an ellipse of minor axis to major axis ratio (b/a) of 0.4 and an arc segment of one quadrant of 85° from posterior-inferior of T12 to posterior-superior of S1 was found to closely approximate (least squares error of 1.2 mm) the average lumbar curvature of 50 healthy subjects (FIG. 7). This project was published in 1998.¹¹ In a follow-up study, the ability of this lumbar elliptical model to discriminate between healthy subjects and low back pain subjects was studied.¹² Here, the lumbar lordosis of four groups of subjects was measured via radiography and subjected to elliptical modeling using a computer iteration process. The four groups included: 50 healthy subjects, 50 acute low back pain subjects free from pathology, 50 chronic low back pain subjects free from pathology, and a group of 24 chronic low back pain subjects with various lumbar degenerative pathologies. In 11/13 measurements we found statistically significant differences between the groups; including elliptical model parameters. Thus our elliptical lumbar model has been found to have predictive validity.

In 2002¹³ and 2003¹⁴, two thoracic spine models (FIG. 8) were published. Both were portions of an ellipse, with an approximate b/a ratio of 0.7 (as compared to the 1998 lumbar b/a ratio of 0.4). As in the CBP® cervical and lumbar modeling projects, we published average and ideal normal values for each thoracic segmental angle and for global angles of kyphosis. All these modeling studies were performed with a computer iteration process, originated by Dr. Tad Janik. This iteration process attempts to pass geometric shapes through the posterior body margins that were digitized on lateral radiographs by Dr. Don Harrison, Dr. Tad Janik, and Dr. Deed Harrison.

In 2004 we have revisited our cervical model. The 1996 cervical model data were obtained from “by-hand” line drawing measurements of lateral cervical radiographs, whereas the lumbar and thoracic modeling was performed with computer iterations, in the least squares sense, from digitized vertebral body corners. We wondered if our recent more mathematical approach would affect our old cervical model. We obtained 266 out of the original (from 1996⁴) 400 subjects and digitized these radiographs. We obtained a circular model very similar to our 1996 result, with some interesting differences. This project is in press for 2004 at Spine.¹⁵ Importantly, in this same study, our cervical circular model was able to discriminate between healthy subjects and neck pain subjects.¹⁵ Here, the cervical lordosis of healthy subjects was compared to acute neck pain and chronic neck pain subjects. For all subjects in each of three groups, subjects were free from significant pathology, did not have segmental or total kyphosis, and had minimal anterior head translation. In this manner, the determination and pain relevance of hypo-lordosis was sought. The x-ray measurements were found to be statistically significant different between the groups; including the circular model parameter or radius of curvature.

This finally leads us to a full spine model that could be a compilation of all past CBP® average normal and ideal spinal models. However, when attempted, the thoracic and lumbar models did not fit properly at T12. We discovered that our 1998 lumbar model, which was derived from subjects in Normal, Ill., had a posterior translation of T12 compared to S1 due to overweight female subjects.¹¹ By way of a literature review, we found that subjects with a body mass index (BMI=weight (Kg)/Height (m)²) in the overweight range, will have a net increase in their lumbar lordosis.^{16,17} Subsequently, we modeled the lumbar spines of 50 normal subjects obtained from Dr. Phil Paulk's clinic in Stockbridge, Ga. with a more normal BMI. These were the same subjects that we had used to derive our thoracic models and thus continuity was found at T12 between the thoracic ellipse and the new lumbar elliptical model (b/a=0.32). This new model is in review at present.¹⁸

Importantly, our new cervical model was an almost perfectly fit at T1 with the T1-S1 model. FIG. 3 is the new full spine model, used as the model overlay on x-rays, and illustrates that there is a near vertical alignment of C1-T1-T12, and S1. Optimal sagittal balance of the cervical, thoracic, and lumbo-pelvic spine is a highly discussed topic in the literature.¹⁹⁻²⁵ An anterior or posterior displaced sagittal balance has been linked to the development of a number of health disorders including: neck pain and upper back pain, low back pain, increased muscle loads, increased stresses on spinal discs, accelerated spinal degeneration, spondylolisthesis, and scoliosis.¹⁹⁻²⁵ Lastly, a circle is a special ellipse (with b/a=radius/radius=1), and thus, the CBP® full spine normal model is composed of separate ellipses for the different spinal region.

The CBP® average normal and Ideal Spinal Model finalized in 2004 is a validated ‘evidence based’ model. This model is useful clinically as an outcome of spinal rehabilitative care, in comparison studies of healthy subjects to different spinal disorder populations, in surgical outcome studies, and in analytical modeling studies to use as an initial starting position of neutral spinal geometry. It should be understood that this model will be tweaked as more research is completed.

### Appendix 2—Dempster's Body Segment Parameter Data for 2-D Studies

The mathematical model used to assist in identifying global subluxations from the adjustments to the positioning of a scalable digital model over the digital images of the lateral, posterior and anterior views of a patient is based on Dempster's Body Segment Parameter Data for 2-D Studies (See FIG. 9).

The actual body segment parameters are identified in Table 3 & 4 from the D. A. Winter, Biomechanics and Motor Control of Human Movement, Second edition. John Wiley & Sons, Inc., Toronto, 1990: (See Table 3 & 4)

The calculations used to digitally position the actual body segment parameters are identified below from the D. A. Winter, Biomechanics and Motor Control of Human Movement, Second edition. John Wiley & Sons, Inc., Toronto, 1990:

$${\sum }_{i = 1}^{n} {P}_{i} = 1.000$$

where n is the number of body segment and i is the segment number and Pᵢ is the segment mass proportion

$${m}_{total body} = {\sum }_{i = 1}^{n} {m}_{i}$$

mᵢ is mass of a segment

Rₚᵣₒₓᵢₘₐₗ+R_{distal}=1.000
R is distance to centre of gravity as proportion of segment length

rₚᵣₒₓᵢₘₐₗ=Rₚᵣₒₓᵢₘₐₗ×length
rₚᵣₒₓᵢₘₐₗ is distance from centre of gravity to proximal end

s_{cg}=sₚᵣₒₓᵢₘₐₗ+Rₚᵣₒₓᵢₘₐₗ(s_{distal}−sₚᵣₒₓᵢₘₐₗ)
s represents position in x, y or z directions

$${s}_{limb} = \frac{{\sum }_{i = 1}^{L} {P}_{i} {s}_{{cg}_{1}}}{{\sum }_{i = 1}^{L} {P}_{i}}$$

here L is the number of segments in the limb

$${s}_{total body} = {\sum }_{i = 1}^{n} {P}_{i} {s}_{{cg}_{i}}$$

kₚᵣₒₓᵢₘₐₗ=Kₚᵣₒₓᵢₘₐₗ×length
kₚᵣₒₓᵢₘₐₗ is radius of gyration for axes through the proximal end and Kₚᵣₒₓᵢₘₐₗ is the radius of gyration as a proportion of the segment length

K_{cg}=\sqrt{Kₚᵣₒₓᵢₘₐₗ²−Rₚᵣₒₓᵢₘₐₗ²}

Kₚᵣₒₓᵢₘₐₗ=\sqrt{K_{cg}²+Rₚᵣₒₓᵢₘₐₗ²}

I_{cg}=m(K_{cg}×length)²
I_{cg} is moment of inertia about an axis through the centre of gravity

Iₚᵣₒₓᵢₘₐₗ=mk_{cg}²+mrₚᵣₒₓᵢₘₐₗ²

Iₚᵣₒₓᵢₘₐₗ=m(K_{cg}×length)²+m(Rₚᵣₒₓᵢₘₐₗ×length)²

$${I}_{total body} = {\sum }_{i = 1}^{n} {I}_{{cg}_{i}} + {\sum }_{i = 1}^{n} {m}_{i} {r}_{i}^{2}$$

where rᵢ is the distance between the total body centre of gravity and each segment's centre of gravity

### REFERENCES

1. Yoganandan et al. Mathematical and finite element analysis of spine injuries. Crit Rev Biomed Eng 1987; 15:29-90.
2. Harrison D D. Class Notes for a 3^{rd} quarter Spinal Biomechanics course. Sunnyvale, Calif.: Northern California College of Chiropractic, 1979.
3. Harrison D D, Janik T J, Troyanovich S J, Harrison D E, Colloca C J. Evaluations of the Assumptions Used to Derive an Ideal Normal Cervical Spine Model. J Manipulative Physiol Ther 1997; 20(4): 246-256.
4. Harrison D D, Janik T J, Troyanovich S J, Holland B. Comparisons of Lordotic Cervical Spine Curvatures to a Theoretical Ideal Model of the Static Sagittal Cervical Spine. Spine 1996; 21(6):667-675.
5. Delmas A Types rachidiens de statique corporelle. Revue de Morphophysiologie, 1951.
6. Goetz H F. Graphic Representation of the curves of the Spinal Column. JAOA 1908; 7(5)
7. Pettibon B R, Loomis. Pettibon Biomechanics (22 articles in a series). Today's Chiropractic. 1973-1975.
8. Harrison D E, Harrison D D, Cailliet R, Troyanovich S J, Janik T J. Cobb Method or Harrison Posterior Tangent Method: Which is Better for Lateral Cervical Analysis? Spine 2000; 25: 2072-78.
9. Harrison D E, Cailliet R, Harrison D D, Janik T J, Holland B. Centroid, Cobb or Harrison Posterior Tangents: Which to Choose for Analysis of Thoracic Kyphosis? Spine 2001; 26(11): E227-E234.
10. Harrison D E, Harrison D D, Janik T J, Harrison S O, Holland B. Determination of Lumbar Lordosis: Cobb Method, Centroidal Method, TRALL or Harrison Posterior Tangents? Spine 2001; 26(11): E236-E242.
11. Janik T J, Harrison D D, Cailliet R, Troyanovich S J, Harrison D E. Can the Sagittal Lumbar Curvature be Closely Approximated by an Ellipse? J Orthop Res 1998; 16(6):766-70.
12. Harrison D D, Cailliet R, Janik T J, Troyanovich S J, Harrison D E, Holland B. Elliptical Modeling of the Sagittal Lumbar Lordosis and Segmental Rotation Angles as a Method to Discriminate Between Normal and Low Back Pain Subjects. J Spinal Disord 1998; 11(5): 430-439.
13. Harrison D E, Janik T J, Harrison D D, Cailliet R, Harmon S. Can the Thoracic Kyphosis be Modeled with a Simple Geometric Shape? The Results of Circular and Elliptical Modeling in 80 Asymptomatic Subjects. J Spinal Disord Tech 2002; 15(3): 213-220.
14. Harrison D D, Harrison D E, Janik T J, Cailliet R, Haas J W. Do Alterations in Vertebral and Disc Dimensions Affect an Elliptical Model of the Thoracic Kyphosis? Spine 2003; 28(5): 463-469.
15. Harrison D D, Harrison D E, Janik T J, Cailliet R, Haas J W, Ferrantelli J, Holland B. Modeling of the Sagittal Cervical Spine as a Method to Discriminate Hypo-Lordosis: Results of Elliptical and Circular Modeling in 72 Asymptomatic Subjects, 52 Acute Neck Pain Subjects, and 70 Chronic Neck Pain Subjects. Spine 2004; in press.
16. Tuzun C, Yorulmaz I, Cindas A, Vata S. Low back pain and posture. Clin Rheumatol 1999; 18:308-312.
17. Ridola C, Palma A, Ridola G, Sanflippo A, Atmasio P L, Zummo G. Changes in the lumbosacral segment of the spine due to overweight in adults. Preliminary remarks. Ital J Anat Embryol 1994; 99:133-143.
18. Harrison D D, Harrison D E, Colloca C J, Cailliet R, Janik T J, Haas J W. Normal Spinal Model from T1 to S1: Results of Elliptical Modeling in 50 Normal Subjects. 2004; in review.
19. Beck A, Killus J. Normal posture of spine determined by mathematical and statistical methods. Aerospace Medicine 1973; 44(11):1277-1281.
20. Jackson R P, McManus A C. Radiographic analysis of sagittal plane alignment and balance in standing volunteers and patients with low back pain matched for age, sex, and size. Spine 1994; 19:1611-1618.
21. Kawakami M, Tamaki T, Ando M, Yamada H, Hashizume H, Yoshida M. Lumbar sagittal balance influences the clinical outcome after decompression and posterolateral spinal fusion for degenerative lumbar spondylolisthesis. Spine 2002; 27:59-64.
22. Kiefer A, Shirazi-Adl A, Parnianpour M. Synergy of the human spine in neutral postures. Eur Spine J 1998; 7:471-479.
23. Kumar M N, Baklanov A, Chopin D. Correlation between sagittal plane changes and adjacent segment degeneration following lumbar spine fusion. Eur Spine J 2001; 10:314-319.
24. Harrison D E, Colloca C J, Keller T S, Harrison D D, Janik T J. Prediction of sagittal plane loads and stresses in the lumbar spine. A comparison of neutral posture and anterior translation of the thoracic cage. Eur Spine J 2004: in press.
25. Ganju A, Ondra S I, Shaffrey C I. Cervical Kyphosis. Techniques in Orthopaedics 2003; 17(3):345-354.

### List of Tables

TABLE 1
The calculations for each of the mathematical model points

| | Global Subluxation | |
|---|---|---|
| Model Point | Component | Formula |
| iHFApX | origFeetApX | (mHaLAKx + mHaRAKx)/2 |
| iHFRLX | origFeetRLatX | mHrRMOx |
| iHFLLX | origFeetLLatX | mHlLMOx |
| iHPaFX | origPelvApWRfeetX | (mHaRUTx + mHaLUTx)/2 |
| iHPrFX | origPelvRLatWRfeetX | (mHrAUTx + mHrRPSx)/2 |
| iHPlFX | origPelvLLatWRfeetX | (mHlAUTx + mHlLPSx)/2 |
| iHTrFY | axisRotThorRLatWRfeetY | mHrT12y |
| iHPvRy | Pelvic Rotation Y axis | ArcSin(Abs(mHrRPBx − mHrLPBx)/iButDs) * 180/iPi |
| iHPvRy | pelvRy | −Arcsin(Abs(mHlRPBx − mHlLPBx)/iButDs) * 180/iPi |
| iHPvRy | pelvRy | 0 |
| iHPSIR | pelvSlantR | Atn((mHrRPSy − mHrRASy)/(mHrRASx − mHrRPSx)) * 180/iPi |
| iHPSIL | pelvSlantL | Atn((mHlLPSy − mHlLASy)/(mHlLPSx − mHlLASx)) * 180/iPi |
| iHPvRx | pelvRx | ((iHPSIR + iHPSIL)/2) − iHPvSI |
| iHPvRz | pelvRz | Atn((mHaLASy − mHaRASy)/((mHaLASx − mHaRASx)/Cos(iHPvRy * iPi/180))) * 180/iPi |
| iHPvTx | pelvTx | iHPaFX − iHFApX |
| iHPvTz | pelvTz | ((iHPrFX − iHFRLX) + (iHFLLX − iHPIFX))/2 |
| iHTFRy | thorRyWRfeet | 0 |
| iHTFRy | thorRyWRfeet | ArcSin((Abs(mHrRSCx − mHrLSCx))/iScaDs) * 180/iPi |
| iHTFRy | thorRyWRfeet | −ArcSin((Abs(mHlRSCx − mHlLSCx))/iScaDs) * 180/iPi |
| iHTPRy | thorRyWRpelv | iHTFRy − iHPvRy |
| iHTFRx | thorRyWRfeet | (((Atn((mHrT2Sx − mHrT12x)/(mHrT2Sy − mHrT12y)) + Atn((mHlT12x − mHlT2Sx)/ |
| | | (mHlT2Sy − mHlT12y))) * 180/iPi)/2) − iHThSl |
| iHTPRx | thorRxWRpelv | iHTFRx − iHPvRx |
| iHTFRz | thorRzWRfeet | Atn((mHaLACy − mHaRACy) * (Cos(iHTFRy * iPi/180))/(mHaLACx − mHaRACx)) * 180/iPi |
| iHTPRz | thorRzWRpelv | iHTFRz − iHPvRz |
| iHTFTx | thorTxWRfeet | (mHaR8Rx + mHaL8Rx)/2 − iHFApX |
| iHTPTx | thorTxWRpelv | iHTFTx − iHPvTx |
| iHcrRx | corrRx | ((((mHrENTy + mHrT12y)/2) − iHTrFY) * Tan(iHTFRx * iPi/180) + (((mHlENTy + mHlT12y)/2) − |
| | | iHTlFY) * Tan(iHTFRx * iPi/180))/2 |
| iHTFTz | thorTzWRfeet | (((((16 * mHrENTx) + (9 * mHrT12x))/25) − iHFRLX + iHFLLX − (((16 * mHlENTx) + (9 * mHlT12x))/ |
| | | 25))/2) − iHcrRx |
| iHTPTz | thorTzWRpelv | iHTFTz − iHPVTz − iHcrRx |
| iHHFRy | headRyWRfeet | 104 * (Abs(mHaEYEx − mHaRERx)/(Abs(mHaRERx − mHaLERx))) − 52 |
| iHHTRy | headRyWRthor | iHHFRy − iHTFRy |
| iHWLry | wallRLatMidY | (mHrRURy + mHrRLRy + mHrLURy + mHrLLRy)/4 |
| iHWLly | wallLLatMidY | (mHlRURy + mHlRLRy + mHlLURy + mHlLLRy)/4 |
| iHcRER | corrR02Y | 155 * (mHrREAy − iHWLry)/(iCamDs − iWalDs) |
| iHcLER | corrL03Y | 155 * (mHlLEAy − iHWLly)/(iCamDs − iWalDs) |
| iHHFRx | headRxWRfeet | ((Atn((mHrREAy − iHcRER − mHrEYEy)/(mHrEYEx − mHrREAx)) * 180/iPi) + (Atn((mHlLEAy − |
| | | iHcLER − mHlEYEy)/(mHlLEAx − mHlEYEx)) * 180/iPi))/2 |
| iHHTRx | headRxWRthor | IHHFRx − iHTFRx |
| iHHFRz | headRzWRfeet | Atn((mHaLERy − mHaRERy) * (Cos(iHHFRy * iPi/180))/(mHaLERx − mHaRERx)) * 180/iPi |
| iHHTRz | headRzWRthor | iHHFRz − iHTFRz |
| iHc1Rz | corrRz1 | Abs((mHaRERy + mHaLERy)/2 − mHaLIPy) * Tan(iHHTRz * iPi/180) |
| iHc2Rz | corrRz2 | (5 * IHHTRz)/15 |
| iHc1Ry | corrRy1 | Sin(2 * iHHFRy * iPi/180) * (iAPrDs * iAPrDs/4)/(2 * (iCamDs − iWalDs)) |
| iHc2Ry | dxCRy | ((mHrREAx − mHrRETx) + (mHlLETx − mHlLEAx))/2 |
| iHc3Ry | corrRy2 | iHc2Ry * Sin(iHHFRy * iPi/180) − Sin(2 * iHHFRy * iPi/180) * (iHc2Ry * iHc2Ry)/ |
| | | (2 * (iCamDs − iWalDs)) |
| iHHFTx | headTxWRfeet | ((mHaRERx + mHaLERx)/2) − iHFApX + iHc1Rz + iHc2Rz + iHc1Ry − iHc3Ry |
| iHHTTa | aa | (−SIN((90 + iHTFRz) * iPi/180)) |
| iHHTTb | bb | Cos((90 + iHTFRz) * iPi/180) |
| iHHTTc | cc | Sin((90 + iHTFRz) * iPi/180) * mHaENTx − Cos((90 + iHTFRz) * iPi/180) * mHaENTy |
| iHHTTx | headTxWRthor | (−(iHHTTa * (mHaRERx + mHaLERx)/2 + iHHTTb * (mHaRERy + mHaLERy)/ |
| | | 2 + iHHTc)/Sqr(iHHTTa * iHHTTa + iHHTTb * iHHTTb)) + iHc1Rz + iHc2Rz + iHc1Ry − iHc3Ry |
| iHHFTz | headTzWRfeet | ((mHrRETx − iHFRLX) + (iHFLLX − mHlLETx))/2 |
| iHHTTz | headTzWRthor | ((mHrRETx − (mHrT2Sx + mHrENTx)/2) + ((mHlT2Sx + mHlENTx)/2 − mHlLETx))/2 |

TABLE 2
Geometric Models

| | Major | | |
|---|---|---|---|
| Author, Year | Assumption | 2nd Assumption | Arc Angle |
| Goetz, 1908⁶ | Arc of Circle | Radius = Length | 57.3° |
| Pettibon & Loomis, 1974⁷ | Arc of Circle | Radius = Chord | 60° |
| Harrison, 1979² | Arc of Circle | H/L = [sin θ]/θ | 63° |
| Harrison et al, 1996⁴ | Arc of Circle | H/L = [sin θ]/θ | 63° |

TABLE 3
Dempster's Body Segment Parameter Data for 2 D studies

| | Endpoints | Seg. mass/ | Centre of mass/ | | Radius of gyration/ | | |
|---|---|---|---|---|---|---|---|
| Segment | (proximal to | total mass | segment length | | segment length | | |
| name | distal) | (P) | (Rₚᵣₒₓᵢₘₐₗ) | (R_{distal}) | (K_{cg}) | (Kₚᵣₒₓᵢₘₐₗ) | (K_{distal}) |
| Hand | wrist axis to | 0.0060 | 0.506 | 0.494 | 0.297 | 0.587 | 0.577 |
| | knuckle II third finger | | | | | | |
| Forearm | elbow axis to | 0.0160 | 0.430 | 0.570 | 0.303 | 0.526 | 0.647 |
| | ulnar styloid | | | | | | |
| Upper | glenohumeral joint to | 0.0280 | 0.436 | 0.564 | 0.322 | 0.542 | 0.645 |
| arm | elbow axis | | | | | | |
| Forearm | elbow axis to | 0.0220 | 0.682 | 0.318 | 0.468 | 0.827 | 0.565 |
| & hand | ulnar styloid | | | | | | |
| Upper | glenohumeral joint to | 0.0500 | 0.530 | 0.470 | 0.368 | 0.645 | 0.596 |
| extremity | elbow axis | | | | | | |
| Foot | lateral malleolus to | 0.0145 | 0.500 | 0.500 | 0.475 | 0.690 | 0.690 |
| | head metatarsal II | | | | | | |
| Leg | femoral condyles to | 0.0465 | 0.433 | 0.567 | 0.302 | 0.528 | 0.643 |
| | medial malleolus | | | | | | |
| Thigh | greater trochanter to | 0.1000 | 0.433 | 0.567 | 0.323 | 0.540 | 0.653 |
| | femoral condyles | | | | | | |
| Leg | femoral condyles to | 0.0610 | 0.606 | 0.394 | 0.416 | 0.735 | 0.572 |
| & foot | medial malleolus | | | | | | |
| Lower | greater trochanter to | 0.1610 | 0.447 | 0.553 | 0.326 | 0.560 | 0.650 |
| extremity | medial malleolus | | | | | | |
| Head | C7-T1 to ear canal | 0.0810 | 1.000 | 0.000 | 0495 | 1.116 | 0.495 |
| Shoulder | sternoclavicular joint to | 0.0158 | 0.712 | 0.288 | | | |
| | glenohumeral joint | | | | | | |
| Thorax | C7-T1 to T12-L1 | 0.2160 | 0.820 | 0.180 | | | |
| Abdomen | T12-L1 to L4-L5 | 0.1390 | 0.440 | 0.560 | | | |

TABLE 4
Dempster's Body Segment Parameter Data for 2 D studies

| | Endpoints | Seg. mass/ | Centre of mass/ | | Radius of gyration/ | | |
|---|---|---|---|---|---|---|---|
| Segment | (proximal to | total mass | segment length | | segment length | | |
| name | distal) | (P) | (Rₚᵣₒₓᵢₘₐₗ) | (R_{distal}) | (K_{cg}) | (Kₚᵣₒₓᵢₘₐₗ) | (K_{distal}) |
| Pelvis | L4-L5 to trochanter | 0.1420 | 0.105 | 0.895 | | | |
| Thorax | C7-T1 to L4-L5 | 0.3550 | 0.630 | 0.370 | | | |
| & abdomen | | | | | | | |
| Abdomen | T12-L1 to | 0.2810 | 0.270 | 0.730 | | | |
| & pelvis | greater trochanter | | | | | | |
| Trunk | greater trochanter to | 0.4970 | 0.495 | 0.505 | 0.406 | 0.640 | 0.648 |
| | glenohumeral joint | | | | | | |
| Trunk | greater trochanter to | 0.5780 | 0.660 | 0.340 | 0.503 | 0.830 | 0.607 |
| & head | glenohumeral joint | | | | | | |
| Head, arms | greater trochanter to | 0.6780 | 0.626 | 0.374 | 0.496 | 0.798 | 0.621 |
| & trunk | glenohumeral joint | | | | | | |
| Head, arms | greater trochanter to | 0.6780 | 1.142 | −0.142 | 0.903 | 1.456 | 0.914 |
| & trunk | midrib | | | | | | |

What is claimed is:

1. A method of acquiring biomechanical segment data and vertebrae positioning data for use in the detection of global subluxations & segment subluxations and a correlation between the two. Specifically the method uses the following steps alone or in combination:

2. Part 1: Modeling the biomechanics of the body using a mathematical model that is superimposed on the patient's body to assist in detecting global Subluxations (postural) by the analysis of head, rib cage, and pelvis postures in three-dimensions (3D) as rotations and translations along the three X, Y, and Z axes.

3. Taking front, side and back view digital images of a patient and automatically superimposing mathematical model which is scalable (based on the individual's height and body type) over the different views.

4. Manually adjusting the computer generated digital mathematical model, as required, by placing adhesive strips on some of the patient's body segments or CBP® eye gear and adjusting the resulting scalable lines to these strips or eye gear.

5. Assist in identifying the global subluxations (postural) and creating an impact assessment and suggest a personalized mirror-imaged exercise routine based on the results.

6. Part 2: Mathematically modeling the vertebrae of the spine by superimposing a scalable digital model over the individual's spine to assist in detecting segment Subluxations (spinal).

7. Superimposing the scalable mathematical vertebral model over the digital images of one or more Spinal X-rays.

8. Manually adjusting the computer generated digital mathematical model, as required, by moving the lines of the model.

9. Assist in identifying the segment Subluxations (spinal) and creating an impact assessment based on the results.

10. Part 3: Correlating the results of the positioning of global subluxations to the segment subluxations of the spine from the use of a third mathematical model (postural and spinal coupling).

11. Assist in validating the global subluxations (postural) and segment subluxations (spinal) coupling and providing the practitioner with discrepancies.

12. Aiding the practitioner in modifying the global subluxations assessment and refining the personalized mirror-imaged exercise routine, if required.

13. A method to assist in identifying global subluxations (postural) and their relative severity in a patient comprising the steps of:

14. Loading digital images of a patient from the anterior, posterior and lateral views.

15. Cropping these images vertically at the top of the patient's head and at the bottom of the feet.

16. Then automatically scaling the image to obtain specific distances based on the patient's height and the aforementioned cropping in claim 16.

17. Cropping these digital images horizontally, based on a ruler applied to the wall behind the individual, and automatically scaling the image.

18. Using the calculated vertical and horizontal distances to superimpose the scalable digital model in the lateral, posterior and anterior views.

19. Calculating vertical and horizontal plumb line using the position of the scalable digital model in the lateral, posterior and anterior views.

20. Manually adjusting the computer generated digital mathematical model, as required, by moving the lines of the model.

21. Calculating the differences in the positioning of the model, in each view, to provide the practitioner with the angle of deviation values and the distance from plumb line values.

22. Using the results of claim 21 in respect to the average or normal values published in the Index Medicus literature to identify the severity of the global subluxations.

23. Based on the results from claim 21 and claim 22 create an impact assessment and suggest a personalized mirror-imaged exercise routine.

24. A method to assist in the detection of segment Subluxations (spinal) and their relative severity in a patient comprising the steps of:

25. Loading digital images of a patient's spinal X-rays.

26. Superimposing a digital mathematical model of the vertebrae of the spine to find the specific points on the digital X-ray images.

27. Manually adjusting the computer generated digital mathematical spinal model, as required, by moving the lines of the model.

28. Using the results from claim 26 and claim 27 the points are analyzed using the digital mathematical model of the vertebrae to arrive at angles and distances.

29. The angles and distances arrived at in claim 28 are compared to published normal values in the Index Medicus literature.

30. An assessment report is generated for the practitioner based on the results for claim 28 and claim 29.

31. A method to assist with correlating the results global subluxations assessment and segment subluxations assessment (postural and spinal coupling).

32. Correlating the results from claim 21 and claim 22 with those from claim 28 and claim 29.

33. Using the postural and spinal coupling model to further assist the practitioner in refining global subluxations (postural) based on the segment subluxations (spinal) analysis.

34. Aiding the practitioner in further refining the personalized mirror-imaged exercise routine, mirror-imaged adjustments on a drop table and mirror-imaged traction.
